(19) United States
(12) Patent Application Publication
(10) Pub. No.: US 2005/0054584 A1
(43) Pub. Date: Mar. 10, 2005

(54) Use of sugar phosphates, sugar phosphate analog, amino acids, amino acid analogs for modulating transaminases and/or the association of p36/ malate dehydrogenase

(76) Inventors: Erich Eigenbrodt, Linden (DE); Sybille Mazurek, Linden (DE); Stefan Mullner, Langenfeld (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/471,866

(22) PCT Filed: Mar. 12, 2002
(86) PCT No.: PCT/DE02/00921

(30) Foreign Application Priority Data
Mar. 13, 2001 (DE) 101 12 925.4

(51) Int. Cl. A61K 31/7024; A61K 31/66; A61K 31/401; A61K 31/198

(52) U.S. Cl. 514/23; 514/561; 514/562; 514/102; 514/423

(57) ABSTRACT

The invention relates to the use of a substance being selected from the group consisting of “sugar phosphates, sugar phosphate analogs, amino acids, amino acid analogs, and mixtures of said substances” for producing a pharmaceutical composition for reducing weight and/or preventing delayed damage caused by diabetes mellitus by modulating the association p36/malate dehydrogenase and/or transaminases.

### FIELD OF THE INVENTION

The invention relates to the use of substances for producing a pharmaceutical composition for modulating transaminases or the p36/malate dehydrogenase complex.

### BACKGROUND OF THE INVENTION

A widespread problem in affluent societies is obesity of many people caused by wrong nutrition. Obesity will lead to diverse health problems, from heart/circulation problems to orthopedic complications. There are the most various approaches for controlling obesity or for reducing weight of obese persons. Virtually all approaches have in common a purely dietetic element. This means that the person within a monitored period of time will take a reduced number of calories and reduce stored fat, if applicable supported by movement therapies. All these approaches have in common that after expiration of the monitored period of time, the person usually will return to the wrong nutrition and other life habits with the consequence of a subsequent weight gain. This has been termed the yo-yo effect.

Diabetes mellitus patients have the additional health problem that in the course of the disease delayed damages in particular in the form of vascular damages will occur. These are irreversible. The reason for the vascular damages is an increased production of peroxides caused by the disease in the metabolization via the malate-aspartate shuttle.

Prior Art.

For instance, from the document Eigenbrodt, E. et al., Biochemical and Molecular Aspects of Selected Cancers, 2:311 ff (1996), various metabolism mechanisms in the cell are known, by means of which glycolytic hydrogen is transported from the cytosol into the mitochondria. These are the glycerol 3-phosphate shuttle, the malate-aspartate shuttle, and the citrate shuttle. In well-differentiated tissues, all three shuttles are active. The glycerol 3-phosphate shuttle is strongly affected by thyroxine. This leads to a strong increase of the energy consumption with thyroid gland hyperactivity. In tumor cells, the glycerol 3-phosphate shuttle is always switched off. Therefore, hydrogen generated within the glycolysis in the glycerin aldehyde 3-phosphate dehydrogenase reaction can be transported either via the glycerol 3-phosphate shuttle of the malate-aspartate shuttle into the mitochondria, where it is burned. In the case of the transport via the glycerol 3-phosphate shuttle, 2 moles ATP per mole hydrogen are generated. In the case of the transport via the malate-aspartate shuttle, 3 moles ATP per mole hydrogen are generated. The latter shuttle thus operates at a higher energy yield. Further, it is known that the malate dehydrogenase as well as transaminases are components of the malate-aspartate shuttle.

From the document Mazurek, S. et al., J. Cell. Physiol. 167:238-250 (1996), it is known in the art that the malate dehydrogenase in a cell exists in three forms, a mitochondrial form comprising the mitochondrial isoenzyme and its forerunner, a cytosolic form and a form in association with the protein p36 (phosphoprotein 36). The latter form is a forerunner of the mitochondrial isoenzyme being held by the association with p36 in the cytosol. The association promotes the hydrogen transport via the malate-aspartate shuttle.

The above findings have been obtained in the prior art regularly with reference to transformation-caused specialties of the cell metabolism in tumor tissue. Other illness references are not addressed.

Technical Object of the Invention.

The invention is based on the technical object to provide active ingredients, which are capable to control the metabolization of food such that obesity is prevented or reduced and delayed damages with diabetes mellitus are prevented.

Basics of the Invention.

For achieving said technical object, the invention teaches the use of a substance selected from the group consisting of “sugar phosphates, sugar phosphate analogs, amino acids, amino acid analogs, and mixtures of said substances” for producing a pharmaceutical composition for reducing weight and/or preventing delayed damage caused by diabetes mellitus by modulating the association p36/malate dehydrogenase and/or transaminases.

The invention is based on one hand on the finding that the cited substances dissolve the association p36/malate dehydrogenase in the cytosol with the consequence that the isoenzyme migrates into the mitochondria, where it is removed from the cytosolic part of the malate-aspartate shuttle. The consequence is that the metabolization of food is driven to the glycerol 3-phosphate shuttles, which on one hand has a smaller energy yield and leads on the other hand to fewer peroxides. Consequently, a given amount of taken food is metabolized less efficiently. The treated person will thus lose weight with unchanged eating habits or will keep a reduced weight. Further, damages by diabetes mellitus—overweight persons are a group having an increased risk of falling ill—are prevented. The invention is further based on the finding that the cited substances simultaneously can inhibit transaminases, i.e. a synergetic effect occurs in the shift to the glycerol 3-phosphate shuttle.

The term analogs designates compounds that can be deducted from the structures of natural amino acids or sugars, i.e. being different therefrom, effecting however the same or an even stronger modulation of the p36/malate dehydrogenase association and/or transaminase inhibition than the basic natural substance. An analog may in particular be a derivative; another not naturally occurring group can replace i.e. a naturally occurring functional group or an H atom. This relates to side chains as well as to the core structure; for instance, a nitrile group may in particular replace the carboxyl group of an amino acid. In the case of the sugar phosphate analogs, a —CN group may replace a phosphate group. It is also possible to replace several phosphate groups by one —CN group each.

### PREFERRED EMBODIMENTS OF THE INVENTION

Various not limiting embodiments of the invention are possible. For instance, a pharmaceutical composition according to the invention may contain several compounds used according to the invention. Further, a pharmaceutical composition according to the invention may contain an active ingredient being different from an active ingredient used according to the invention. Then it is a combination preparation. The various used active ingredients may be prepared in a single dosage form, i.e. the active ingredients are mixed in the dosage form. It is however also possible to prepare the various active ingredients in spatially separated dosage forms of identical or different type.

A combination preparation may for instance be used in the insulin therapy for diabetes mellitus. Therein, the insulin is prepared in a mixture with one or more compounds used according to the invention for the IM application. It is understood that the compound used therein should in the case of IM application be well tolerated by the tissue, should in particular not cause any tissue irritation.

For the indication according to the invention for reducing weight, the used compound may also be used as a food supplement. Therein special dietetic foods are produced, to which is added the compound or the compounds in defined concentrations. The selection of such compounds is desirable, which are as neutral in taste as possible.

With regard to the active ingredient used according to the invention it is possible that the substance is selected from the group consisting of “serine, cycloserine, valine, leucine, isoleucine, proline, methionine, cysteine, amino isobutyrate, aminooxyacetate, CHBA, fructose-1,6-bisphosphate, glycerate-2,3-bisphosphate, glycerate-3-phosphate, ribose-1,5-bisphosphate, ribulose-1,5-bisphosphate and mixtures of such substances. CHBA is 2-cyano-3-hydroxy-but-2-(4-trifluoromethyl-phenyl)-amide.”

Preferably, the substance is selected from the group consisting of compounds of the formula I and mixtures of such compounds.

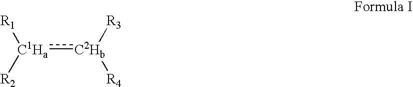

wherein a and b may be identical or different and are 0 or 1,

wherein R1=—H, C1-C18 alkyl, cycloalkyl or aryl,

wherein R2=—H, C1-C18 alkyl, cycloalkyl or aryl, C1-C8 hydroxyalkyl, C1-C8 mercaptoalkyl, C1-C8 ether, C1-C8 thioether, C1-C8 aminoalkyl, with C1-C8 alkyl, cycloalkyl or phenyl, —CONHX2 or —CNHNHX2 N-substituted C1-C8 aminoalkyl, with -Hal and/or —OX1 substituted aryl, —OX1, —SX1, —COO⁻, —(CH₂)ₙ—COOX1 or —COOX1 with X1=—H, C1-C18 alkyl, cycloalkyl or aryl, and with n=1-8,

wherein R3=—CN, —C═N—X2, —COO⁻, —COOX2, —CO—X2, —CO—NHX2 with X2=—H, C1-C18 alkyl, cycloalkyl or aryl,

wherein R4=—H, —O—P, ═O, aryl, —NHY or —CO—NHZ with Y=—H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or CaₒHalₘ and/or —N—CO—CaₒHalₘ and/or C1-C8 alkyl, cycloalkyl or aryl substituted phenyl or with -Hal and/or —O-Hal and/or C1-C8 alkyl, cycloalkyl or aryl substituted naphthyl (Hal=—F, —Cl, or —Br), wherein m=1-3 and o=3-m,

wherein a and b correspond to the number of remaining carbon valences at C¹ and C²,

wherein via R3 a ring connection to C¹ under elimination of X1 in R2 and X2 in R3 may be provided.

Particularly preferred substances are characterized by that

a=1 and b=0,

R1=—H,

R2=—H, C1-C18 alkyl, cycloalkyl or aryl, C1-C8 hydroxyalkyl, C1-C8 mercaptoalkyl, C1-C8 aminoalkyl, N-substituted C1-C8 aminoalkyl, substituted aryl, —OX1, —SX1,

R3=—CN,

R4=═O.

These particularly preferred substances are typically 2 or α-oxonitriles. These substances are amino acid analogs with high efficiency.

Hₐ may also be replaced by —SH or —SR2. By the ring connection via R3, a homo or heteroatomic aromatic ring comprising C1 and C2 may also be formed, which carries further substituents from the groups described above, for instance homo or heteroatomic aromatic rings (substituted or not substituted).

It has to be noted, with regard to cycloalkyl and aryl groups, that hereby homo as well as heteroatomic aromatic groups are covered. Examples for heterocyclic groups are: furanyl, thiophenyl, pyrrolyl, isopyrrolyl, 3-isopyrrolyl, pyrazolyl, 2-isoimidazolyl, triazolyl, oxazolyl, isooxzolyl, thiazolyl, isothiazolyl, pyridyl, pyrazinyl, pyrimidinyl, pyridazinyl, piperazinyl, triazinyl, oxazinyl, indenyl, benzofuranyl, benzothiofuranyl, indolyl, isoindazolyl, benzoxazolyl, and the mentioned groups may be in part hydrated. The mentioned groups may also be formed by a ring structure with inclusion of C1 in the ring. In the following, specific compounds 1 to 42 are stated.

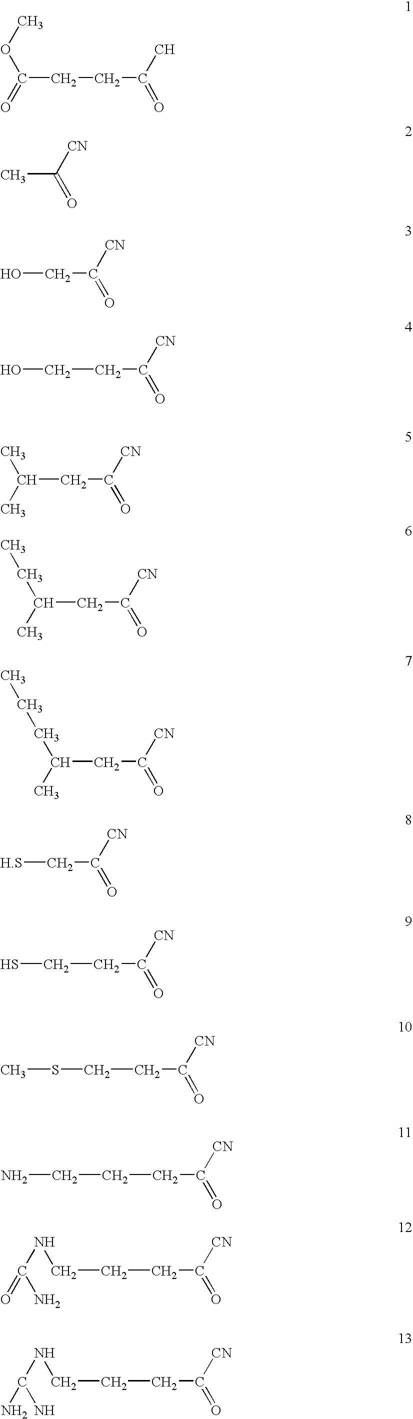

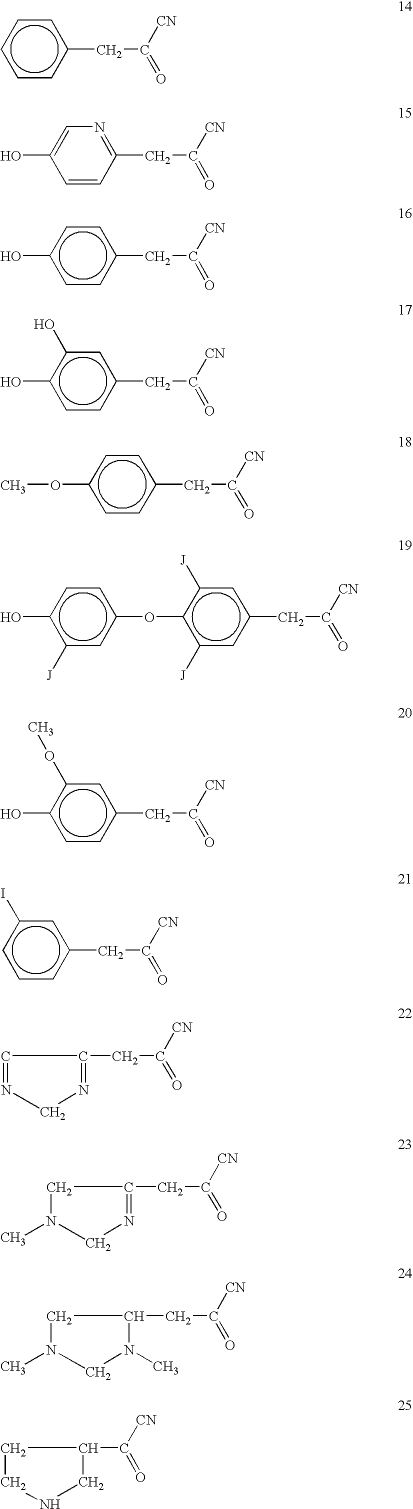

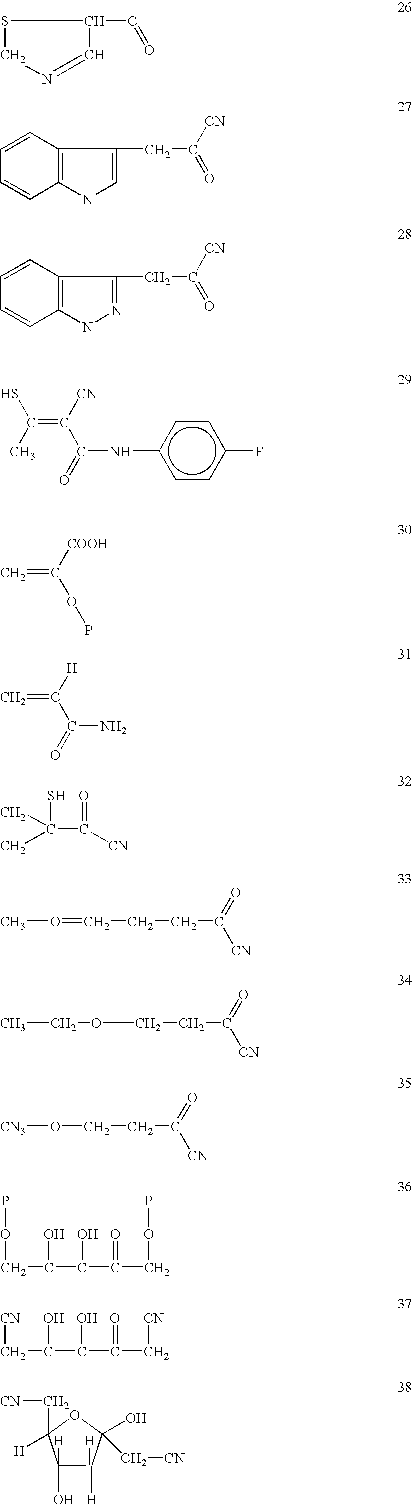

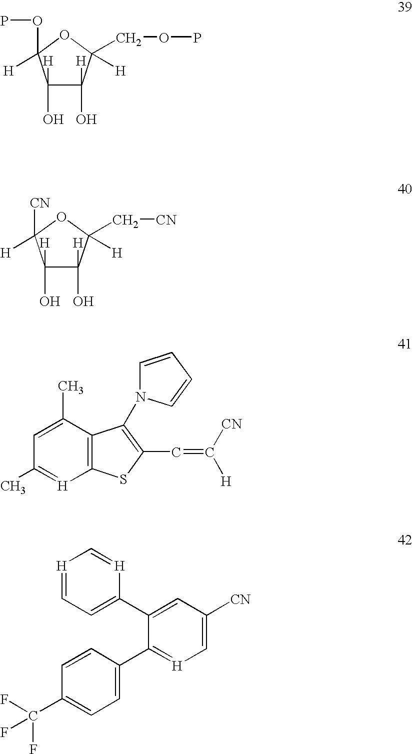

As counter ions for ionic compounds according to formula I can be used Na⁺, K⁺, Li⁺ or cyclohexylammonium.

The drugs produced with the compounds according to the invention may be administered in an oral, intramuscular, periarticular, intraarticular, intravenous, intraperitoneal, subcutaneous, or rectal manner. Particularly preferred, however, is the intravenous administration, in particular in the case of CHBA or aminooxyacetate (NH₂—CO—COOH), or the oral administration.

The invention also relates to a method for preparing a drug which is characterized by that at least one compound used according to the invention is brought into contact with a pharmaceutically suitable and physiologically well tolerated carrier and if applicable mixed with further suitable active ingredients, additional or auxiliary substances and prepared to the desired dosage form.

Finally, the invention teaches a combination preparation for treating diabetes mellitus containing insulin and one or more compounds used according to the invention as well as a dietetic food containing one or more compounds used according to the invention.

Suitable solid or liquid galenic dosage forms are for instance granulates, powders, dragées, tablets, (micro) capsules, suppositories, syrups, juices, suspensions, emulsions, drops or injectable solutions as well as preparations with protracted release of the active ingredient, for the preparation of which usual means such as carrier substances, explosion, binding, coating, swelling, sliding or lubricating agents, flavoring substances, sweeteners and solution mediators are used.

Auxiliary substances are for instance magnesium carbonate, titanium dioxide, lactose, mannite and other sugars, talcum, milk protein, gelatin, starch, cellulose and its derivatives, animal and plant oils such as cod-liver oil, sunflower, peanut or sesame oil, polyethylene glycols and solvents, such as sterile water and one or poly-valent alcohols, e.g. glycerin.

Preferably, the drugs are prepared and administered in dosage units, each unit containing as an active component a defined dose of the compound according to formula I of the invention. With solid dosage units such as tablets, capsules, dragées or suppositories, this dose may be 1 to 5,000 mg, preferably 50 to 1,000 mg, and for injection solutions in an ampoule form 0.3 to 300 mg, preferably 10 to 100 mg.

For treating an adult patient of 50 to 100 kg weight, for instance 70 kg, for instance daily doses of 20 to 5,000 mg active ingredient, preferably 100 to 500 mg, are indicated. Under certain circumstances, higher or lower daily doses may be recommendable. The administration of the daily dose may be a one-off administration in the form of a single dosage unit or several smaller dosage units as well as a multi-administration of separated doses in certain intervals.

In the following, the invention is explained in more detail with reference to examples representing embodiments only.

### EXAMPLE 1

### Agents for Reducing Weight

A mixture of aminooxyacetate and conventional galenic auxiliary substances are pressed so to form tablets, the amounts of the components being selected such that a tablet contains 750 mg aminooxyacetate.

A person to be treated (70 kg) takes one of the above tablets with liquid before every eating (3 times per day). Eating takes place according to the usual habits of the person.

Within a period of treatment of 4 weeks, a weight curve of the persons treated according to the invention is obtained, which corresponds to the weight curve of a control group, which has taken approx. 15 to 20% less food than the pro-band group.

### EXAMPLE 2

### Dietetic Food

A so-called medium-fat margarine from vegetable fats, water and usual emulgators and auxiliary substances and dyes was prepared. 2.5 weight parts aminooxyacetate were homogeneously mixed to 100 weight parts margarine, and the obtained mixture was subjected to final wrapping.

### EXAMPLE 3

### Combination Preparation for Treating Diabetes Mellitus

A usual insulin injection solution is prepared, and 0.25 weight parts aminooxyacetate were mixed to 10 weight parts of this solution and packed in ampoules in usual insulin doses. A patient is injected IM with the thus obtained combination preparation according to the treatment plan set up for this specific patient.

1. A method for reducing weight of a subject and/or preventing delayed damage caused by diabetes mellitus by modulating the association p36/malate dehydrogenase and/or transaminases, comprising administering a pharmaceutical composition comprising a substance selected from the group consisting of sugar phosphates, sugar phosphate analogs, amino acids, amino acid analogs, and mixtures of said substances.

2. The method of claim 1, wherein the substance is selected from the group consisting of serine, cycloserine, valine, leucine, isoleucine, proline, methionine, cysteine, amino isobutyrate, aminooxyacetate, CHBA, fructose-1,6-bisphosphate, glycerate-2,3-bisphosphate, glycerate-3-phosphate, ribose-1,5-bisphosphate, ribulose-1,5-bisphosphate, analogs of such substances and mixtures of such substances.

3. The method of claim 1, wherein the substance is selected from the group consisting of compounds of the formula I and of mixtures of such compounds, 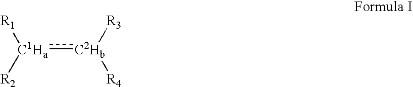
wherein a and b may be identical or different and are 0 or 1,
wherein R1=—H, C1-C18 alkyl, cycloalkyl or aryl,
wherein R2=—H, C1-C18 alkyl, cycloalkyl or aryl, C1-C8 hydroxyalkyl, C1-C8 mercaptoalkyl, C1-C8 ether, C1-C8 thioether, C1-C8 aminoalkyl, with C1-C8 alkyl, cycloalkyl or phenyl, —CONHX2 or —CNHNHX2 N-substituted C1-C8 aminoalkyl, with -Hal and/or —OX1 substituted aryl, —OX1, —SX1, —COO⁻, —(CH₂)ₙ—COOX1 or —COOX1 with X1=—H, C1-C18 alkyl, cycloalkyl or aryl, and with n=1-8,
wherein R3=—CN, —C═N—X2, —COO⁻, —COOX2, —CO—X2, —CO—NHX2 with X2=—H, C1-C18 alkyl, cycloalkyl or aryl,
wherein R4=—H, —O—P, ═O, aryl, —NHY or —CO—NHZ with Y=—H, —CO—R (R=C1-C18 alkyl, cycloalkyl or aryl or —NHA, with A=H or C1-C18 alkyl, cycloalkyl or aryl), and Z=phenyl, naphthyl, with -Hal and/or —O-Hal and/or CAₒHalₘ and/or —N—CO—CAₒHalₘ and/or C1-C8 alkyl, cycloalkyl or aryl substituted phenyl or with -Hal and/or —O-Hal and/or C1-C8 alkyl, cycloalkyl or aryl substituted naphthyl (Hal=—F, —Cl, or —Br), wherein m=1-3 and o=3-m,
wherein a and b correspond to the number of remaining carbon valences at C¹ and C²,
wherein via R3 a ring connection to C¹ under elimination of X1 in R2 and X2 in R3 may be provided.

4. The method of claim 3, wherein
a=1 and b=0,
R1=—H,
R2=—H, C1-C18 alkyl, cycloalkyl or aryl, C1-C8 hydroxyalkyl, C1-C8 mercaptoalkyl, C1-C8 aminoalkyl, N-substituted C1-C8 aminoalkyl, substituted aryl, —OX1, —SX1,
R3=CN,
R4=═O.

5. The method of claims 1 to 4, wherein the pharmaceutical composition is prepared for intravenous application.

6. The method of claims 1 to 4, wherein the pharmaceutical composition is prepared for an administration of a daily dose of 0.1 to 20 mg per kg body weight.

7. A pharmaceutical composition containing insulin and at least one substance according to one of claims 1 to 4, wherein the ratio of the substances to insulin (w/w) is in the range of 0.001:100 to 10:100.

8. A dietetic food containing one or more substances according to one of claims 1 to 4, wherein 0.01 to 10 weight parts of the substance are mixed to 100 weight parts food.

9. The composition of claim 7, wherein the ratio is in the range of 0.01:100 to 3:100.

10. The composition of claim 7, wherein the ratio is in the range of 0.1:100 to 0.3:100.

11. The composition of claim 7, wherein the composition is prepared for intravenous application.

12. The composition of claim 7, wherein the composition is prepared for an administration of a daily dose of 0.1 to 20 mg per kg body weight.

13. The composition of claim 7, wherein the composition is prepared for intravenous administration of a daily dose of 0.1 to 20 mg per kg body weight.

14. The food of claim 8, wherein 0.1 to 10 weight parts of the substance are mixed to 100 weight parts food.

15. The food of claim 8, wherein 1 to 3 weight parts of the substance are mixed to 100 weight parts food.

16. The method of claim 5, wherein the pharmaceutical composition is prepared for an administration of a daily dose of 0.1 to 20 mg per kg body weight.
